SEQUENCE LISTING (3 sequences)

SEQ ID NO: 1 - DNA, 22 bp, Artificial Sequence
  InfA forward primer
gaccratcctgtcacctctgac

SEQ ID NO: 2 - DNA, 24 bp, Artificial Sequence
  InfA reverse primer
agggcattytggacaaakcgtcta

SEQ ID NO: 3 - DNA, 20 bp, Artificial Sequence
  InfA fluorescent probe
cacaaatcctaaaattccct

(19) United States
(12) Patent Application Publication
(10) Pub. No.: US 2017/0275682 A1
(43) Pub. Date: Sep. 28, 2017

(54) NUCLEIC ACID AMPLIFICATION REACTION METHOD, NUCLEIC ACID AMPLIFICATION REACTION APPARATUS, AND NUCLEIC ACID AMPLIFICATION REACTION REAGENT

(71) Applicant: Seiko Epson Corporation, Tokyo (JP)

(72) Inventor: Masayuki UEHARA, Matsumoto (JP)

(21) Appl. No.: 15/467,402

(22) Filed: Mar. 23, 2017

(30) Foreign Application Priority Data
Mar. 28, 2016 (JP) 2016-064468

(51) Int. Cl. C12Q 1/68; B01L 7/00

(52) U.S. Cl. CPC C12Q 1/6848; C12Q 1/686; B01L 7/525; B01L 2300/18; B01L 2200/14

(57) ABSTRACT

A nucleic acid amplification reaction method includes subjecting a reaction mixture containing a nucleic acid amplification reaction reagent to be used for amplifying a nucleic acid to a thermal cycle for amplifying the nucleic acid, wherein in the thermal cycle, a heating time for an annealing reaction and an elongation reaction is 1 sec or more and 10 sec or less, the nucleic acid amplification reaction reagent contains a forward primer, a reverse primer, a polymerase, and a fluorescently labeled probe, the concentration of the forward primer is 0.4 μM or more and 3.2 μM or less, the concentration of the reverse primer is 0.4 μM or more and 3.2 μM or less, the amount of the polymerase is 0.5 U or more and 4 U or less, and the concentration of the fluorescently labeled probe is 0.15 μM or more and 1.2 μM or less.

This application claims the benefit of Japanese Patent Application No. 2016-064468, filed on Mar. 28, 2016. The content of the aforementioned application is incorporated herein by reference in its entirety.

### BACKGROUND

1. Technical Field

The present invention relates to a nucleic acid amplification reaction method, a nucleic acid amplification reaction apparatus, and a nucleic acid amplification reaction reagent.

2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agricultural and livestock industries. As technologies for utilizing genes, technologies such as a PCR (polymerase chain reaction) method are widely used. Nowadays, the PCR method has become an indispensable technology for elucidation of information on biological materials.

The PCR method is a method of amplifying a target nucleic acid by subjecting a solution (reaction mixture) containing a nucleic acid to be amplified (target nucleic acid) and a reagent to a thermal cycle. The thermal cycle is a treatment of periodically subjecting the reaction mixture to two or more temperature steps. In the PCR method, a method of performing a two- or three-step thermal cycle is generally performed.

For example, JP-A-2012-115208 (Patent Document 1) describes a nucleic acid amplification reaction apparatus in which a reaction container loaded with a reaction mixture (liquid droplet) and a liquid (such as an oil) immiscible with the reaction mixture and having a smaller specific gravity than the reaction mixture is rotated around the axis of rotation so as to move the reaction mixture due to the difference in specific gravity, whereby a thermal cycle is performed. In Patent Document 1, a fluorescently labeled probe is used for detecting whether a nucleic acid is amplified.

Meanwhile, a production time for a product by PCR has been required to be reduced. However, in a technique described in Patent Document 1, when a PCR reaction time (a time for a denaturation reaction or a time for an annealing reaction/an elongation reaction) is reduced (when PCR is accelerated), for example, a nucleic acid and the fluorescently labeled probe do not sufficiently hybridize to each other, and the amplification of the nucleic acid cannot be detected with high sensitivity in some cases.

### SUMMARY

An advantage of some aspects of the invention is to provide a nucleic acid amplification reaction method capable of detecting the amplification of a nucleic acid with high sensitivity even if PCR is accelerated. Another advantage of some aspects of the invention is to provide a nucleic acid amplification reaction apparatus capable of detecting the amplification of a nucleic acid with high sensitivity even if PCR is accelerated. Still another advantage of some aspects of the invention is to provide a nucleic acid amplification reaction reagent capable of detecting the amplification of a nucleic acid with high sensitivity even if PCR is accelerated.

A nucleic acid amplification reaction method according to an aspect of the invention includes subjecting a reaction mixture containing a nucleic acid amplification reaction reagent to be used for amplifying a nucleic acid to a thermal cycle for amplifying the nucleic acid, wherein in the thermal cycle, a heating time for an annealing reaction and an elongation reaction is 1 sec or more and 10 sec or less, the nucleic acid amplification reaction reagent contains a forward primer, a reverse primer, a polymerase, and a fluorescently labeled probe, the concentration of the forward primer contained in the reaction mixture is 0.4 μM or more and 3.2 μM or less, the concentration of the reverse primer contained in the reaction mixture is 0.4 μM or more and 3.2 μM or less, the amount of the polymerase contained in the reaction mixture is 0.5 U or more and 4 U or less, and the concentration of the fluorescently labeled probe contained in the reaction mixture is 0.15 μM or more and 1.2 μM or less.

According to such a nucleic acid amplification reaction method, the amplification of a nucleic acid can be detected with high sensitivity even if PCR is accelerated (see the below-mentioned “4. Experimental Example” for the details).

In the nucleic acid amplification reaction method according to the aspect of the invention, it is preferred that the nucleic acid amplification reaction reagent contains dNTPs, and the concentration of the dNTPs contained in the reaction mixture is 0.125 mM or more and 1 mM or less.

According to such a nucleic acid amplification reaction method, the amplification of a nucleic acid can be more reliably detected with high sensitivity even if PCR is accelerated.

In the nucleic acid amplification reaction method according to the aspect of the invention, it is preferred that the concentration of the forward primer is 0.8 μM or more and 3.2 μM or less, the concentration of the reverse primer is 0.8 μM or more and 3.2 μM or less, the amount of the polymerase is 1 U or more and 4 U or less, the concentration of the fluorescently labeled probe is 0.3 μM or more and 1.2 μM or less, and the concentration of the dNTPs is 0.25 mM or more and 1 mM or less.

According to such a nucleic acid amplification reaction method, the amplification of a nucleic acid can be detected with higher sensitivity even if PCR is accelerated.

In the nucleic acid amplification reaction method according to the aspect of the invention, it is preferred that the concentration of the forward primer is 1.6 μM or more and 3.2 μM or less, the concentration of the reverse primer is 1.6 μM or more and 3.2 μM or less, the amount of the polymerase is 2 U or more and 4 U or less, the concentration of the fluorescently labeled probe is 0.6 μM or more and 1.2 μM or less, and the concentration of the dNTPs is 0.5 mM or more and 1 mM or less.

According to such a nucleic acid amplification reaction method, the amplification of a nucleic acid can be detected with much higher sensitivity even if PCR is accelerated.

In the nucleic acid amplification reaction method according to the aspect of the invention, it is preferred that the concentration of the forward primer is 2.4 μM or more and 3.2 μM or less, the concentration of the reverse primer is 2.4 μM or more and 3.2 μM or less, the amount of the polymerase is 3 U or more and 4 U or less, the concentration of the fluorescently labeled probe is 0.9 μM or more and 1.2 μM or less, and the concentration of the dNTPs is 0.75 mM or more and 1 mM or less.

According to such a nucleic acid amplification reaction method, the amplification of a nucleic acid can be detected with even much higher sensitivity even if PCR is accelerated.

A nucleic acid amplification reaction apparatus according to an aspect of the invention includes a mounting portion capable of mounting a nucleic acid amplification reaction cartridge which holds a reaction mixture containing a nucleic acid amplification reaction reagent to be used for amplifying a nucleic acid and a liquid having a different specific gravity from the reaction mixture and immiscible with the reaction mixture, and has a flow channel through which the reaction mixture moves, a temperature gradient forming portion which forms a temperature gradient in the flow channel, and a moving mechanism which moves the reaction mixture so as to subject the reaction mixture to a thermal cycle for amplifying the nucleic acid, wherein the moving mechanism moves the reaction mixture so that a heating time for an annealing reaction and an elongation reaction in the thermal cycle is 1 sec or more and 10 sec or less, the nucleic acid amplification reaction reagent contains a forward primer, a reverse primer, a polymerase, and a fluorescently labeled probe, the concentration of the forward primer contained in the reaction mixture is 0.4 μM or more and 3.2 μM or less, the concentration of the reverse primer contained in the reaction mixture is 0.4 μM or more and 3.2 μM or less, the amount of the polymerase contained in the reaction mixture is 0.5 U or more and 4 U or less, and the concentration of the fluorescently labeled probe contained in the reaction mixture is 0.15 μM or more and 1.2 μM or less.

According to such a nucleic acid amplification reaction apparatus, the amplification of a nucleic acid can be detected with high sensitivity even if PCR is accelerated.

A nucleic acid amplification reaction reagent according to an aspect of the invention is a nucleic acid amplification reaction reagent which is contained in a reaction mixture to be subjected to a thermal cycle for amplifying a nucleic acid, wherein in the thermal cycle, a heating time for an annealing reaction and an elongation reaction is 1 sec or more and 10 sec or less, the nucleic acid amplification reaction reagent contains a forward primer, a reverse primer, a polymerase, and a fluorescently labeled probe, the concentration of the forward primer contained in the reaction mixture is 0.4 μM or more and 3.2 μM or less, the concentration of the reverse primer contained in the reaction mixture is 0.4 μM or more and 3.2 μM or less, the amount of the polymerase contained in the reaction mixture is 0.5 U or more and 4 U or less, and the concentration of the fluorescently labeled probe contained in the reaction mixture is 0.15 μM or more and 1.2 μM or less.

According to such a nucleic acid amplification reaction reagent, the amplification of a nucleic acid can be detected with high sensitivity even if PCR is accelerated.

### BRIEF DESCRIPTION OF THE DRAWINGS

The invention will be described with reference to the accompanying drawings, wherein like numbers reference like elements.

FIG. 1 is a cross-sectional view schematically showing a nucleic acid amplification reaction cartridge according to an embodiment.

FIG. 2 is a cross-sectional view schematically showing a nucleic acid amplification reaction cartridge according to an embodiment.

FIG. 3 is a cross-sectional view schematically showing a nucleic acid amplification reaction cartridge according to an embodiment.

FIG. 4 is a cross-sectional view schematically showing a nucleic acid amplification reaction cartridge according to an embodiment.

FIG. 5 is a perspective view schematically showing a nucleic acid amplification reaction apparatus according to an embodiment.

FIG. 6 is a perspective view schematically showing a nucleic acid amplification reaction apparatus according to an embodiment.

FIG. 7 is an exploded perspective view schematically showing a nucleic acid amplification reaction apparatus according to an embodiment.

FIG. 8 is a cross-sectional view schematically showing a nucleic acid amplification reaction apparatus according to an embodiment.

FIG. 9 is a cross-sectional view schematically showing a nucleic acid amplification reaction apparatus according to an embodiment.

FIG. 10 is a functional block diagram of a nucleic acid amplification reaction apparatus according to an embodiment.

FIG. 11 is a flowchart for illustrating a nucleic acid amplification reaction method according to an embodiment.

FIG. 12 is a graph showing the results of PCR in the case where the concentration of a nucleic acid amplification reaction reagent was changed.

FIG. 13 is a graph showing the results of PCR in the case where the concentration of a nucleic acid amplification reaction reagent was changed.

FIG. 14 is a graph showing the results of PCR under standard conditions and high-speed conditions.

FIG. 15 is a graph showing the results of multiplex PCR.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, all the configurations described below are not necessarily essential components of the invention.

### 1. Nucleic Acid Amplification Reaction Reagent

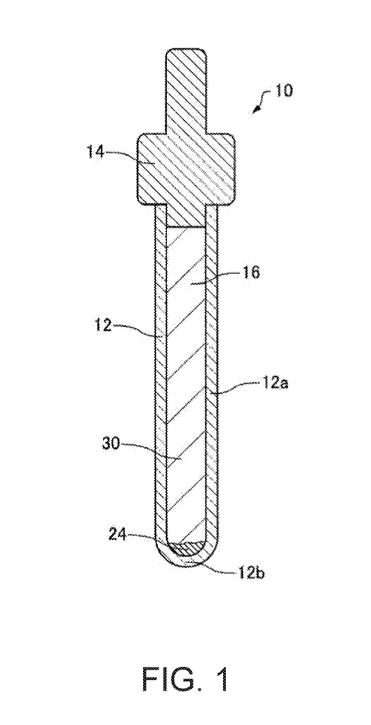

First, a nucleic acid amplification reaction reagent according to this embodiment will be described. The nucleic acid amplification reaction reagent is held in a nucleic acid amplification reaction cartridge. FIG. 1 is across-sectional view schematically showing a nucleic acid amplification reaction cartridge 10 according to this embodiment.

As shown in FIG. 1, the nucleic acid amplification reaction cartridge 10 includes a container 12 and a cap 14.

As shown in FIG. 1, the container 12 has, for example, a side wall portion 12a in a cylindrical shape and a bottom portion 12b in a semispherical shape. The container 12 has a flow channel 16. The flow channel 16 is formed by the container 12. The flow channel 16 extends along the central axis (not shown) of the side wall portion 12a in a cylindrical shape. The cap 14 closes the opening of an end portion facing the bottom portion 12b of the container 12. The cap 14 can be attached to and detached from the container 12. The material of the container 12 and the cap 14 is, for example, a glass, a polymer, a metal, or the like.

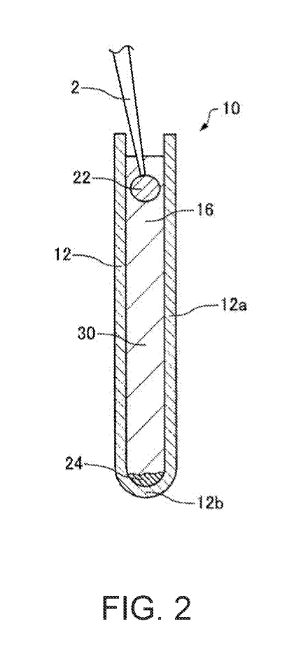

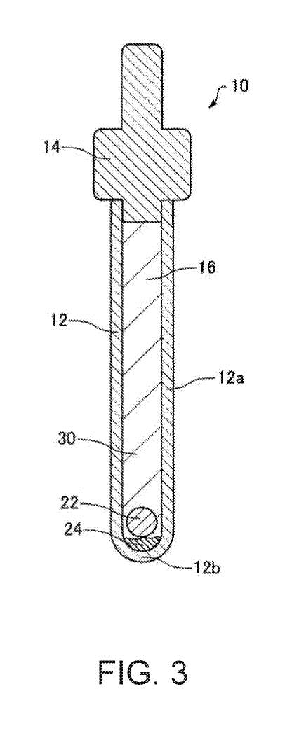

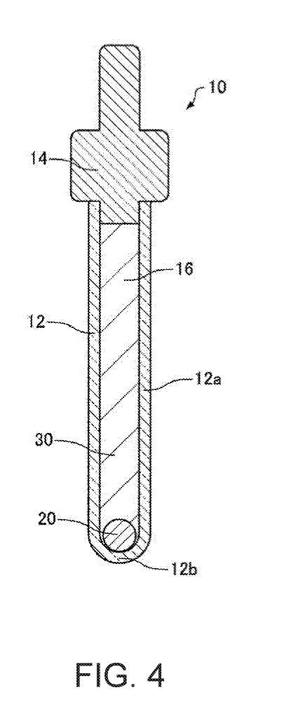

To the bottom portion 12b of the container 12, for example, a nucleic acid amplification reaction reagent 24 in a lyophilized state is fixed. When the cap 14 is detached and a template nucleic acid solution 22 is introduced into such a nucleic acid amplification reaction cartridge 10 using a pipette 2 or the like as shown in FIG. 2, the introduced template nucleic acid solution 22 sinks to the bottom portion 12b as shown in FIG. 3 and comes into contact with the nucleic acid amplification reaction reagent 24. The nucleic acid amplification reaction reagent 24 in a lyophilized state is dissolved in the aqueous component of the template nucleic acid solution 22 and incorporated into the template nucleic acid solution 22 so as to form a reaction mixture 20 as shown in FIG. 4. Therefore, the reaction mixture 20 contains the template nucleic acid and the nucleic acid amplification reaction reagent 24, and thus serves as a place for allowing a nucleic acid amplification reaction to proceed.

The reaction mixture 20 is held in the nucleic acid amplification reaction cartridge 10 and placed in the flow channel 16. The reaction mixture 20 is maintained in a state of a liquid droplet in a liquid 30. In the example shown in the drawing, the shape of the reaction mixture 20 is a sphere. The reaction mixture 20 has, for example, a larger specific gravity than the liquid 30. The reaction mixture 20 moves relatively to the vessel 12 in the flow channel 16 accompanying the movement of the nucleic acid amplification reaction cartridge 10.

The template nucleic acid solution 22 is a solution containing a template nucleic acid. When the template nucleic acid solution 22 is introduced into the container 12, the cap 14 is detached from the container 12, and after the template nucleic acid solution 22 is introduced, the cap 14 is attached to the container 12 again.

The template nucleic acid solution 22 is obtained by, for example, as follows. That is, a specimen, for example, a cell derived from an organism such as a human or a bacterium, a virus, or the like is collected using a collecting tool such as a cotton swab, and a template nucleic acid is extracted from the specimen using a known extraction method. Thereafter, a template nucleic acid solution is purified using a known purification method to have a predetermined concentration. The aqueous component of the template nucleic acid solution 22 is, for example, water (distilled water or sterile water) or a Tris-EDTA (ethylenediaminetetraacetic acid) (TE) solution.

The nucleic acid amplification reaction reagent 24 is held in the nucleic acid amplification reaction cartridge 10 and is placed, for example, in the bottom portion 12b of the container 12 in a lyophilized (freeze-dried) state. The nucleic acid amplification reaction reagent 24 is a reagent to be used for an amplification reaction of a nucleic acid (target nucleic acid). The nucleic acid amplification reaction reagent 24 contains primers, a polymerase, a fluorescently labeled probe, and dNTPs.

The primers are designed to anneal to a target nucleic acid. The nucleic acid amplification reaction reagent 24 contains a forward primer which anneals to one template nucleic acid having a single-stranded structure (single-stranded DNA) after a template nucleic acid having a double-stranded structure (double-stranded DNA) is denatured, and a reverse primer which anneals to the other single-stranded DNA.

The concentration of the forward primer contained in the reaction mixture 20 is 0.4 μM or more and 3.2 μM or less, preferably 0.8 μM or more and 3.2 μM or less, more preferably 1.6 μM or more and 3.2 μM or less, further more preferably 2.4 μM or more and 3.2 μM or less. The concentration of the reverse primer contained in the reaction mixture 20 is, for example, 0.4 μM or more and 3.2 μM or less, preferably 0.8 μM or more and 3.2 μM or less, more preferably 1.6 μM or more and 3.2 μM or less, further more preferably 2.4 μM or more and 3.2 μM or less. When the concentrations thereof are within the above ranges, the amplification of a nucleic acid can be detected with high sensitivity even if PCR is accelerated (see “4. Experimental Example” for the details). The concentration of the forward primer and the concentration of the reverse primer contained in the reaction mixture 20 are, for example, the same.

The polymerase is not particularly limited, however, examples thereof include a DNA (deoxyribonucleic acid) polymerase. The DNA polymerase polymerizes nucleotides complementary to the bases of the template nucleic acid at the end of the primer annealing to the template nucleic acid having a single-stranded structure (single-stranded DNA). The DNA polymerase is preferably a heat-resistant enzyme or an enzyme for PCR, and there are a large number of commercially available products, for example, Taq polymerase, Tfi polymerase, Tth polymerase, modified forms thereof, and the like, however, a hot start DNA polymerase is preferred.

The amount of the polymerase contained in the reaction mixture 20 is 0.5 U or more and 4 U or less, preferably 1 U or more and 4 U or less, more preferably 2 U or more and 4 U or less, further more preferably 3 U or more and 4 U or less. When the amount thereof is within the above range, the amplification of a nucleic acid can be detected with high sensitivity even if PCR is accelerated.

The fluorescently labeled probe is used for quantitatively determining the amplification amount of a nucleic acid. The fluorescently labeled probe is, for example, a hydrolysis probe containing a reporter dye and a quencher dye. The fluorescently labeled probe which is a hydrolysis probe is configured such that the light emission of the reporter dye is suppressed by the quencher dye (by the quenching effect) in close proximity to the reporter dye while the fluorescently labeled probe hybridizes (anneals) to the single-stranded DNA to form a double-stranded structure. However, when the fluorescently labeled probe is degraded by the polymerase, the quenching effect is cancelled, and therefore, the reporter dye emits light. By this light emission, the amplification amount of a nucleic acid can be quantitatively determined.

The concentration of the fluorescently labeled probe contained in the reaction mixture 20 is 0.15 μM or more and 1.2 μM or less, preferably 0.3 μM or more and 1.2 μM or less, more preferably 0.6 μM or more and 1.2 μM or less, further more preferably 0.9 μM or more and 1.2 μM or less. When the concentration thereof is within the above range, the amplification of a nucleic acid can be detected with high sensitivity even if PCR is accelerated (see “4. Experimental Example” for the details).

The term “dNTPs” refers to a mixture of four types of deoxyribonucleotide triphosphates. That is, the term “dNTPs” refers to a mixture of dATP (deoxyadenosine triphosphate), dCTP (deoxycytidine triphosphate), dGTP (deoxyguanosine triphosphate), and dTTP (thymidine triphosphate). The DNA polymerase forms a new DNA by joining dATP, dCTP, dGTP, or dTTP to the end of the primer annealing to the template nucleic acid.

The concentration of the dNTPs contained in the reaction mixture 20 is 0.125 mM or more and 1 mM or less, preferably 0.25 mM or more and 1 mM or less, more preferably 0.5 mM or more and 1 mM or less, further more preferably 0.75 mM or more and 1 mM or less. When the concentration thereof is within the above range, the amplification of a nucleic acid can be detected with high sensitivity even if PCR is accelerated (see “4. Experimental Example” for the details).

The reaction mixture 20 may further contain water or a buffer (for example, a buffer obtained by mixing MgCl₂, Tris-HCl, and KCl).

The liquid 30 is held in the nucleic acid amplification reaction cartridge 10 and is placed in the flow channel 16. In the example shown in the drawing, the flow channel 16 is loaded (filled) with the reaction mixture 20 and the liquid 30. The liquid 30 is a liquid which is immiscible or does not mix with the reaction mixture 20. The liquid 30 is also immiscible with the template nucleic acid solution 22 and the nucleic acid amplification reaction reagent 24. The liquid 30 has a different specific gravity from the reaction mixture 20. Specifically, the liquid 30 has a smaller specific gravity than the reaction mixture 20. Therefore, the reaction mixture 20 moves in a direction in which gravity acts by the action of gravity. The liquid 30 is, for example, a dimethyl silicone oil, a paraffin oil, or the like.

In the above description, an example in which the nucleic acid amplification reaction reagent 24 in a lyophilized state is fixed to the bottom portion 12b of the container 12, and the template nucleic acid solution 22 is introduced into the container 12 to bring the template nucleic acid solution 22 into contact with the nucleic acid amplification reaction reagent 24, whereby the reaction mixture 20 is formed has been described. However, the reaction mixture 20 may be placed in the container 12 by preparing a solution containing the template nucleic acid and the nucleic acid amplification reaction reagent 24 outside the container 12 and introducing the solution into the container 12 loaded with the liquid 30.

### 2. Nucleic Acid Amplification Reaction Apparatus

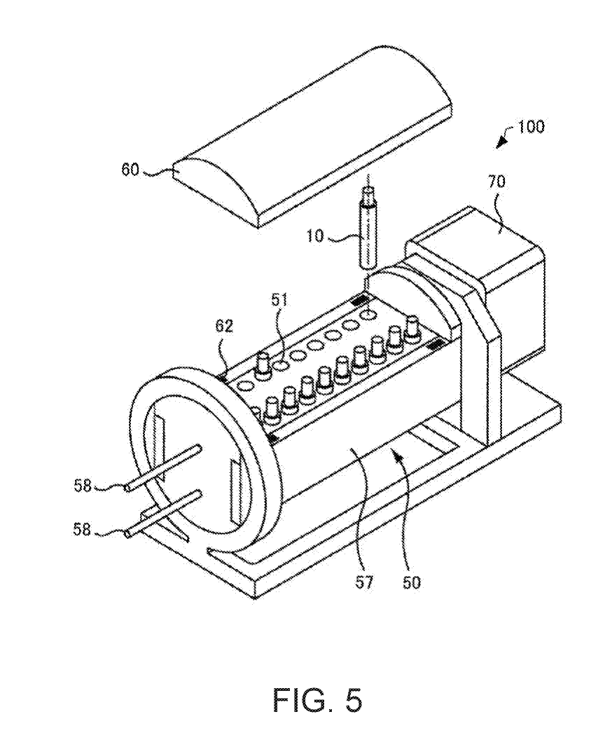

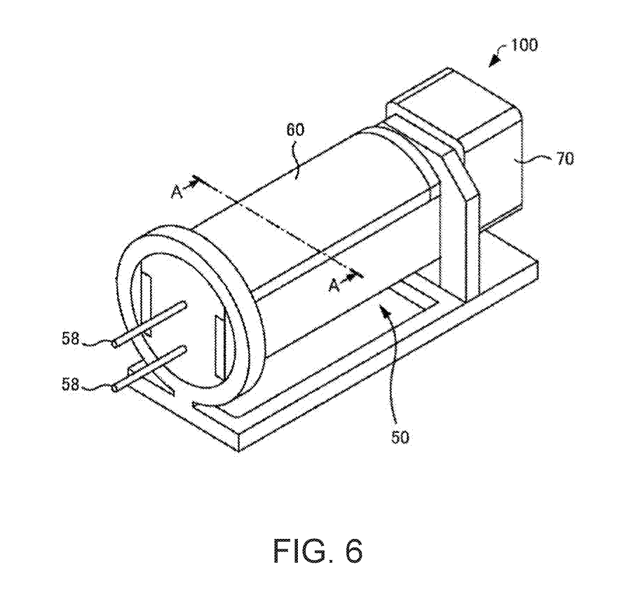

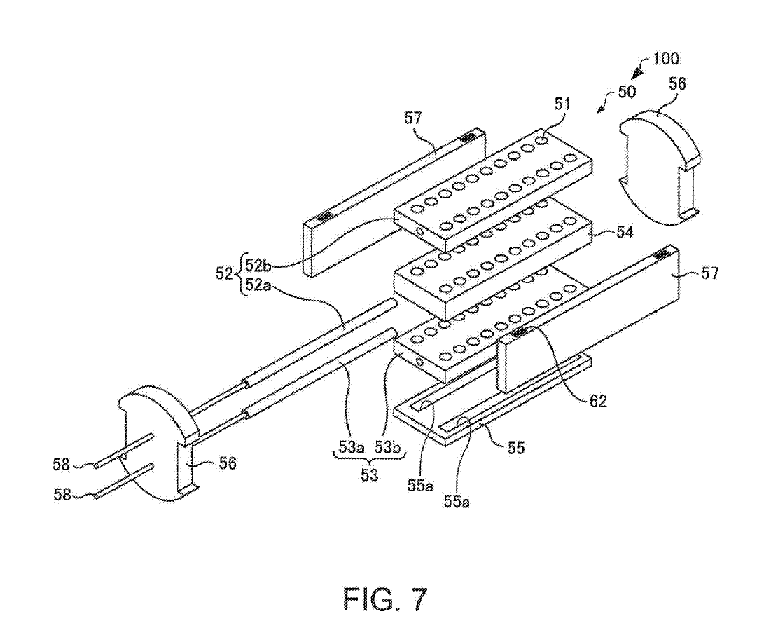

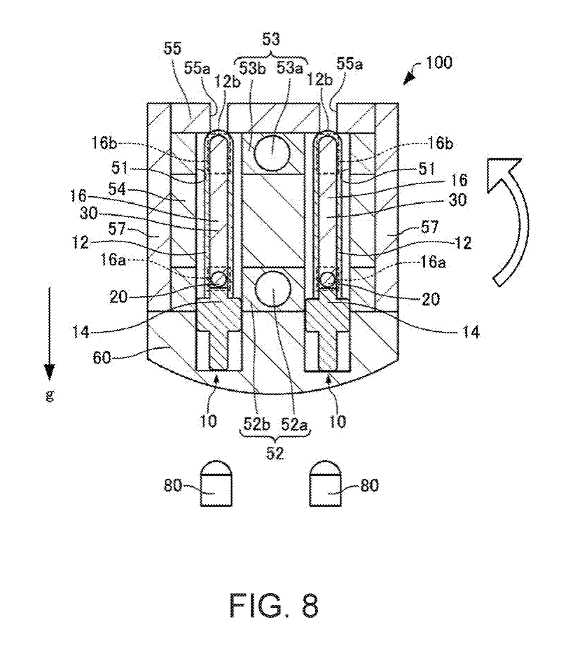

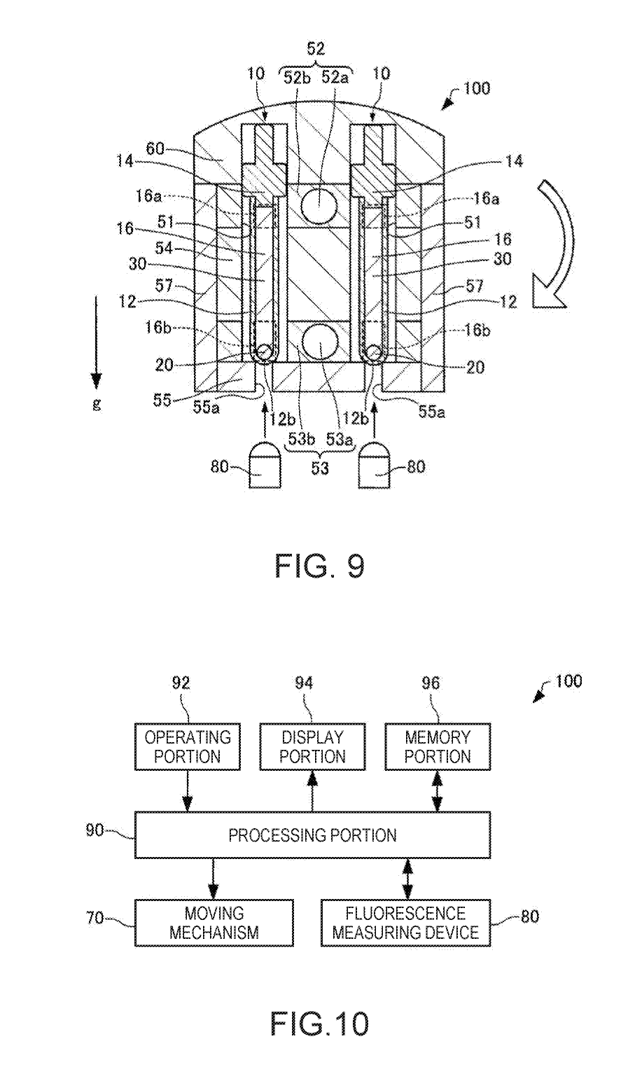

Next, a nucleic acid amplification reaction apparatus according to this embodiment will be described with reference to the drawings. FIGS. 5 and 6 are each a perspective view schematically showing a nucleic acid amplification reaction apparatus 100 according to this embodiment, and FIG. 5 shows a state where a lid body 60 is opened and FIG. 6 shows a state where the lid body 60 is closed. FIG. 7 is an exploded perspective view schematically showing a main body portion 50 of the nucleic acid amplification reaction apparatus 100 according to this embodiment. FIGS. 8 and 9 are each a cross-sectional view taken along the line A-A in FIG. 6 schematically showing the nucleic acid amplification reaction apparatus 100 according to this embodiment. FIG. 10 is a functional block diagram of the nucleic acid amplification reaction apparatus 100 according to this embodiment.

Incidentally, in FIG. 5, the illustration of the nucleic acid amplification reaction cartridge 10 is simplified for the sake of convenience. Further, in FIGS. 5 and 6, the illustration of a fluorescence measuring device 80 is omitted. Further, in FIGS. 8 and 9, the direction of the arrow g is a direction in which gravity acts (gravitational direction). FIG. 8 shows a state where the arrangement of heating portions 52 and 53 is a first arrangement, and FIG. 9 shows a state where the arrangement of the heating portions 52 and 53 is a second arrangement. Further, in FIG. 10, the illustration of the main body portion 50 and the lid body 60 is omitted for the sake of convenience.

As shown in FIGS. 5 to 10, the nucleic acid amplification reaction apparatus 100 includes the main body portion 50, the lid body 60, a moving mechanism 70, the fluorescence measuring device 80, a processing portion 90, an operating portion 92, a display portion 94, and a memory portion 96. The nucleic acid amplification reaction apparatus 100 is, for example, an up-and-down type PCR apparatus.

As shown in FIG. 7, the main body portion 50 has a mounting portion 51, a first heating portion 52, a second heating portion 53, a spacer 54, a bottom plate 55, a flange 56, and a fixing plate 57.

The mounting portion 51 has a structure capable of mounting the nucleic acid amplification reaction cartridge 10. Specifically, as shown in FIG. 5, the mounting portion 51 has a structure in which the nucleic acid amplification reaction cartridge 10 is mounted by plugging (inserting) the cartridge therein. In the example shown in FIG. 7, the mounting portion 51 is a through-hole penetrating a first heat block 52b of the first heating portion 52, the spacer 54, and a second heat block 53b of the second heating portion 53. The number of mounting portions 51 may be two or more, and is 20 in the example shown in the drawing.

In the case where the nucleic acid amplification reaction cartridge 10 is mounted in the mounting portion 51, the first heating portion 52 heats a first region 16a of the flow channel 16 to a first temperature as shown in FIGS. 8 and 9. In the example shown in the drawings, the first heating portion 52 is located closer to the lid body 60 than the second heating portion 53.

The first heating portion 52 has, for example, a mechanism which generates heat and a member which transfers the generated heat to the nucleic acid amplification reaction cartridge 10. In the example shown in FIG. 7, the first heating portion 52 has a first heater 52a and the first heat block 52b. The first heater 52a is, for example, a cartridge heater and is connected to an external power supply (not shown) through a conductive wire 58. The first heater 52a is inserted into a hole provided in the first heat block 52b, and by the heat generated by the first heater 52a, the first heat block 52b is heated. The first heat block 52b is a member which transfers heat generated from the first heater 52a to the nucleic acid amplification reaction cartridge 10. The first heat block 52b is a block made of, for example, aluminum. The first region 16a of the flow channel 16 is a region surrounded by the first heat block 52b.

In the case where the nucleic acid amplification reaction cartridge 10 is mounted in the mounting portion 51, the second heating portion 53 heats a second region 16b of the flow channel 16 to a second temperature which is different from the first temperature. The second heating portion 53 has a second heater 53a and the second heat block 53b. The second heating portion 53 has the same structure and function as the first heating portion 52 except that the region of the nucleic acid amplification reaction cartridge 10 to be heated and the heating temperature. The second region 16b of the flow channel 16 is a region surrounded by the second heat block 53b.

The temperatures of the first heating portion 52 and the second heating portion 53 are controlled by a temperature sensor (for example, a thermocouple) not shown in the drawing and the processing portion 90. For example, by controlling the temperature of the first heating portion 52 to the first temperature and the temperature of the second heating portion 53 to the second temperature, the first region 16a of the nucleic acid amplification reaction cartridge 10 can be heated to the first temperature, and the second region 16b can be heated to the second temperature.

The spacer 54 is provided between the first heating portion 52 and the second heating portion 53. The spacer 54 has a function to thermally insulate the first heating portion 52 from the second heating portion 53.

The bottom plate 55 is a member which holds the nucleic acid amplification reaction cartridge 10. The bottom plate 55 determines the position in the height direction of the nucleic acid amplification reaction cartridge 10. That is, by inserting the nucleic acid amplification reaction cartridge 10 to a position where the nucleic acid amplification reaction cartridge 10 comes into contact with the bottom plate 55, the nucleic acid amplification reaction cartridge 10 can be held at a predetermined position with respect to the heating portions 52 and 53. The bottom plate 55 is provided with a through-hole 55a for allowing excitation light from the fluorescence measuring device 80 and fluorescence of the reaction mixture 20 to pass therethrough.

The flange 56 and the fixing plate 57 are members for fixing the heating portions 52 and 53 and the spacer 54. In the example shown in the drawing, two fixing plates 57 are fitted in the flange 56, and the heating portions 52 and 53, the spacer 54, and the bottom plate 55 are fixed to the fixing plates 57.

The lid body 60 covers the mounting portion 51. In the example shown in FIG. 5, the fixing plate 57 is provided with a magnet portion 62, and the lid body 60 can be fixed to the main body portion 50 by the magnet portion 62. The material of the spacer 54, the bottom plate 55, the flange 56, the fixing plate 57, and the lid body 60 is, for example, a thermal insulating material.

The moving mechanism 70 is a mechanism for rotating the main body portion 50 based on an input signal from the processing portion 90. According to this, the moving mechanism 70 can change the arrangement of the heating portions 52 and 53 between the first arrangement (see FIG. 8) and the second arrangement (see FIG. 9). As a result, the moving mechanism 70 can move the reaction mixture 20 so that the reaction mixture 20 is subjected to a thermal cycle for amplifying the nucleic acid. In the first arrangement, the first heating portion 52 is located on the lower side in the gravitational direction than the second heating portion 53. In the second arrangement, the second heating portion 53 is located on the lower side in the gravitational direction than the first heating portion 52. The moving mechanism 70 has, for example, a motor (not shown) and a drive shaft (not shown). The drive shaft is connected to the main body portion 50 and the flange 56. The drive shaft is provided perpendicularly to the longitudinal direction of the nucleic acid amplification reaction cartridge 10, and when the motor is operated, the main body portion 50 is rotated around the drive shaft as the axis of rotation.

The fluorescence measuring device 80 is a measuring device which measures the fluorescence intensity (fluorescence brightness) of the reaction mixture 20 held in the nucleic acid amplification reaction cartridge 10. The fluorescence measuring device 80 is placed facing the bottom portion 12b of the nucleic acid amplification reaction cartridge 10 at a predetermined distance therefrom in the second arrangement as shown in FIG. 9. The fluorescence measuring device 80 irradiates the reaction mixture 20 with excitation light corresponding to the fluorescent dye of the fluorescently labeled probe contained in the reaction mixture 20 based on the input signal from the processing portion 90 and measures the fluorescence intensity emitted from the reaction mixture 20. The fluorescence measuring device 80 may measure the fluorescence intensity corresponding to one fluorescent dye or may measure the fluorescence intensities corresponding to a plurality of fluorescent dyes.

As shown in FIG. 10, the processing portion 90 performs a process for controlling the moving mechanism 70 or the fluorescence measuring device 80 according to, for example, a program stored in the memory portion 96. The processing portion 90 is realized by, for example, a processor such as a CPU (central processing unit).

The operating portion 92 acquires an operation signal corresponding to the operation by a user and performs a process of sending the operation signal to the processing portion 90. The operating portion 92 is realized by, for example, a button, a key, a touch panel display, a microphone, or the like.

The display portion 94 displays an image formed by the processing portion 90. The display portion 94 is realized by, for example, an LCD (liquid crystal display), a CRT (cathode ray tube), or the like.

The memory portion 96 stores a program, data, or the like for the processing portion 90 to perform various calculation processes or control processes. Further, the memory portion 96 is used as a working region of the processing portion 90, and is also used for temporarily storing the results of calculation performed by the processing portion 90 according to various programs or the like. The memory portion 96 is realized by, for example, an RAM (random access memory) or the like.

### 3. Nucleic Acid Amplification Reaction Method

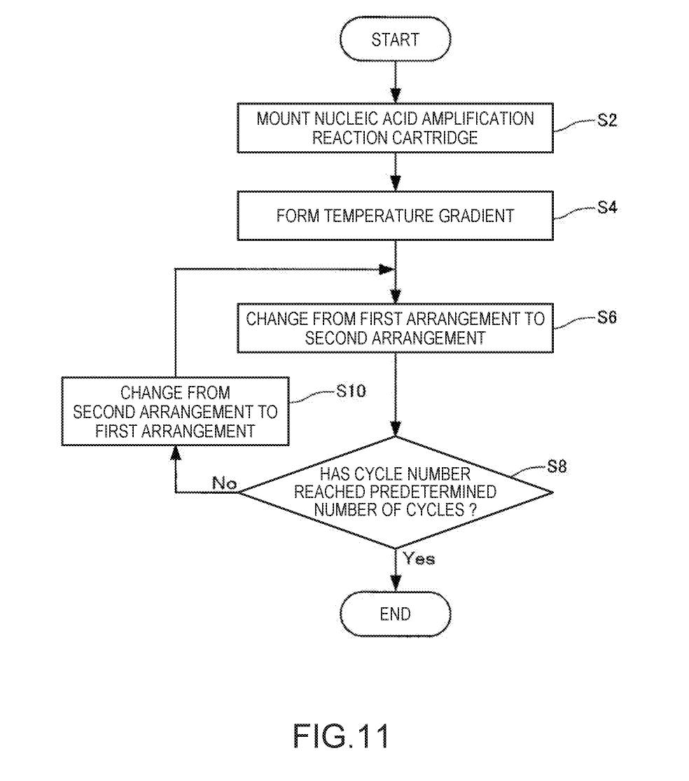

Next, a nucleic acid amplification reaction method according to this embodiment will be described with reference to the drawing. FIG. 11 is a flowchart for illustrating the nucleic acid amplification reaction method according to this embodiment. Hereinafter, as one example, a nucleic acid amplification reaction method using the nucleic acid amplification reaction apparatus 100 will be described.

First, the nucleic acid amplification reaction cartridge 10 is mounted in the mounting portion 51 of the nucleic acid amplification reaction apparatus 100 (Step S2). Specifically, after the reaction mixture 20 is introduced into the container 12 loaded with the liquid 30, the nucleic acid amplification reaction cartridge 10 is mounted in the mounting portion 51. For example, when the nucleic acid amplification reaction cartridge 10 is mounted in the mounting portion 51, the mounting portion 51 is covered with the lid body 60.

Here, the arrangement of the heating portions 52 and 53 is the first arrangement as shown in FIG. 8. In the first arrangement, the first region 16a is located in the lowermost portion of the flow channel 16 in the gravitational direction. Therefore, the reaction mixture 20 having a larger specific gravity than the liquid 30 is located in the first region 16a.

Subsequently, when receiving a signal to start a thermal cycling treatment from the operating portion 92, the processing portion 90 controls the heating portions 52 and 53 to heat the first region 16a and the second region 16b of the nucleic acid amplification reaction cartridge 10 and form a temperature gradient in the flow channel 16 (Step S4). Specifically, the first heating portion 52 heats the first region 16a to a first temperature, and the second heating portion 53 heats the second region 16b to a second temperature which is lower than the first temperature. By doing this, a temperature gradient in which the temperature gradually changes between the first temperature and the second temperature is formed between the first region 16a and the second region 16b of the flow channel 16. Here, the heating portions 52 and 53 are temperature gradient forming portions which form a temperature gradient in which the temperature decreases from the first region 16a to the second region 16b.

The first temperature is a temperature suitable for the dissociation (denaturation reaction) of a double-stranded DNA, and is for example 95° C. or higher and 110° C. or lower, and the second temperature is a temperature suitable for an annealing reaction and an elongation reaction, and is for example 50° C. or higher and 75° C. or lower. The arrangement of the heating portions 52 and 53 is the first arrangement, and therefore, when the nucleic acid amplification reaction cartridge 10 is heated, the reaction mixture 20 is heated to the first temperature.

Subsequently, the processing portion 90 controls the moving mechanism 70 to change the arrangement of the heating portions 52 and 53 from the first arrangement to the second arrangement after a first time (first period) has passed from when the temperature of the first heating portion 52 has reached the first temperature (Step S6). The processing portion 90 may incorporate a timer therein. The first time is a heating time for the denaturation reaction, and is for example 1 sec or more and 10 sec or less. The moving mechanism 70 is controlled by the processing portion 90 to move the reaction mixture 20 so that the first time becomes 1 sec or more and 10 sec or less. Specifically, the processing portion 90 controls the moving mechanism 70 to rotate the main body portion 50 by 180°. By doing this, the arrangement of the heating portions 52 and 53 is changed from the first arrangement to the second arrangement.

As shown in FIG. 9, the second arrangement is an arrangement in which the second region 16b is located in the lowermost portion of the flow channel 16 in the gravitational direction. In the second arrangement, the positional relationship in the gravitational direction between the first region 16a and the second region 16b is opposite to that in the first arrangement. Due to this, the reaction mixture 20 moves from the first region 16a to the second region 16b by the action of gravity. The processing portion 90 stops the operation of the moving mechanism 70 for a second time (second period) after changing the arrangement of the heating portions 52 and 53 to the second arrangement. By doing this, the heating portions 52 and 53 are held in the second arrangement. The second time is a heating time for the annealing reaction and the elongation reaction, and is for example 1 sec or more and 10 sec or less. The moving mechanism 70 is controlled by the processing portion 90 to move the reaction mixture 20 so that the second time becomes 1 sec or more and 10 sec or less.

Subsequently, the processing portion 90 determines whether or not the number of cycles of change from the first arrangement to the second arrangement (cycle number) has reached a predetermined number of cycles previously stored in the memory portion 96 (Step S8). The processing portion 90 stores the cycle number in the memory portion 96 every time when the change from the first arrangement to the second arrangement is performed and compares the cycle number with the predetermined number of cycles previously stored in the memory portion 96.

In the case where the processing portion 90 determines that the cycle number has reached the predetermined number of cycles in Step S8 (“Yes” in FIG. 11), the processing portion 90 completes the process.

On the other hand, in the case where the processing portion 90 determines that the cycle number has not reached the predetermined number of cycles in Step S8 (“No” in FIG. 11), the processing portion 90 allows the process to proceed to Step S10. In Step S10, the processing portion 90 controls the moving mechanism 70 to change the arrangement of the heating portions 52 and 53 from the second arrangement to the first arrangement. The processing portion 90 stops the operation of the moving mechanism 70 for the first time after changing the arrangement of the heating portions 52 and 53 to the first arrangement.

Then, the processing portion 90 allows the process to proceed to Step S6 again, and changes the arrangement of the heating portions 52 and 53 from the first arrangement to the second arrangement.

As described above, the processing portion 90 allows the reaction mixture 20 to reciprocate in the flow channel 16 by rotating the heating portions 52 and 53 (main body portion 50) while changing the position between the first arrangement and the second arrangement until the cycle number reaches the predetermined number of cycles. By doing this, the nucleic acid amplification reaction apparatus 100 can subject the reaction mixture 20 to a thermal cycle for amplifying a nucleic acid.

The processing portion 90 further performs an amplification analysis treatment at the same time as the above-mentioned thermal cycling treatment. By doing this, in the nucleic acid amplification reaction apparatus 100, real-time PCR can be performed. Specifically, the processing portion 90 inputs a signal for giving a measurement instruction to the fluorescence measuring device 80 every time when the heating portions 52 and 53 are held in the second arrangement. Then, the processing portion 90 acquires a fluorescence intensity from the fluorescence measuring device 80 as the measurement result by the fluorescence measuring device 80 and stores the fluorescence intensity in the memory portion 96.

Further, the processing portion 90 may read out the fluorescence intensities for the number of cycles set as the cycle number to be repeated based on the signal input from the operating portion 92 and form an amplification curve showing the transition of fluorescence intensity with respect to the cycle number based on the fluorescence intensities. The processing portion 90 may determine whether the amplification efficiency of the nucleic acid is accepted or rejected based on the amplification curve and display the determination result or the amplification curve on the display portion 94.

In the above description, in Step S8, the processing portion 90 determines whether or not the cycle number has reached the predetermined number of cycles, however, the processing portion 90 may determine whether or not the obtained fluorescence intensity has reached a predetermined value previously stored in the memory portion 96. Then, in the case where the processing portion 90 determines that the obtained fluorescence intensity has reached the predetermined value, the processing portion 90 may complete the process, and in the case where the processing portion 90 determines that the obtained fluorescence intensity has not reached the predetermined value, the processing portion 90 may allow the process to proceed to Step S10.

Further, in the above description, the second temperature is set to a temperature for the annealing reaction and the elongation reaction, however, the second temperature may be set to a temperature for either one of the annealing reaction and the elongation reaction, and the second time may be set as a heating time for either one of the annealing reaction and the elongation reaction. In such a case, although not shown in the drawing, the nucleic acid amplification reaction apparatus 100 has a third heating portion which heats a third region (a region which is different from the first region and the second region) of the flow channel 16 to a third temperature (a temperature which is different from the first temperature and the second temperature). The third temperature is a temperature for the other reaction of the annealing reaction and the elongation reaction, and the third time is a heating time for the other reaction of the annealing reaction and the elongation reaction. However, in order to accelerate PCR, it is preferred to set the second temperature to a temperature for the annealing reaction and the elongation reaction.

### 4. Experimental Example

Hereinafter, the invention will be more specifically described by showing experimental examples below. However, the invention is by no means limited to the following experimental examples.

### 4.1. Preparation of Sample

As a template nucleic acid, 100 copies (1 reaction tube) of a plasmid DNA of a type A influenza (InfA) virus were used. Incidentally, the 100 copies are the LOD (limit of detection) of the LAMP method (Eiken Chemical Co., Ltd.). The template nucleic acid was added to a nucleic acid amplification reaction reagent, whereby the following mixed reagent solutions were prepared. Specifically, seven types of mixed reagent solutions: 1×, 2×, 4×, 6×, 8×, 10×, and 12× were prepared. For example, the “2×” mixed reagent solution contains a polymerase, dNTPs, primers, and a fluorescently labeled probe in amounts twice as high as those of the “1×” mixed reagent solution.

Incidentally, the amount (volume) of a liquid of each reagent below is a value with respect to 10 μL of the mixed reagent. That is, for example, in the “1×” mixed reagent solution, the polymerase is contained in an amount of 0.1 μL in 10 μL of the mixed reagent solution.

### Composition of “1×” Mixed Reagent Solution

| | | |
|---|---|---|
| Platinum Taq polymerase | 0.1 | μL |
| dNTPs (10 mM) | 0.125 | μL |
| Buffer | 2.0 | μL |
| Forward primer for detection of influenza (20 μM) | 0.2 | μL |
| Reverse primer for detection of influenza (20 μM) | 0.2 | μL |
| Fluorescently labeled probe for detection of influenza | 0.15 | μL |
| (10 μM) | | |
| Influenza plasmid DNA (1000 copies) | 0.625 | μL |
| Water | up to 10 | μL |

### Composition of “2×” Mixed Reagent Solution

| | | |
|---|---|---|
| Platinum Taq polymerase | 0.2 | μL |
| dNTPs (10 mM) | 0.25 | μL |
| Buffer | 2.0 | μL |
| Forward primer for detection of influenza (20 μM) | 0.4 | μL |
| Reverse primer for detection of influenza (20 μM) | 0.4 | μL |
| Fluorescently labeled probe for detection of influenza | 0.3 | μL |
| (10 μM) | | |
| Influenza plasmid DNA (1000 copies) | 0.625 | μL |
| Water | up to 10 | μL |

### Composition of “4×” Mixed Reagent Solution

| | | |
|---|---|---|
| Platinum Taq polymerase | 0.4 | μL |
| dNTPs (10 mM) | 0.5 | μL |
| Buffer | 2.0 | μL |
| Forward primer for detection of influenza (20 μM) | 0.8 | μL |
| Reverse primer for detection of influenza (20 μM) | 0.8 | μL |
| Fluorescently labeled probe for detection of influenza | 0.6 | μL |
| (10 μM) | | |
| Influenza plasmid DNA (1000 copies) | 0.625 | μL |
| Water | up to 10 | μL |

### Composition of “6×” Mixed Reagent Solution

| | | |
|---|---|---|
| Platinum Taq polymerase | 0.6 | μL |
| dNTPs (10 mM) | 0.75 | μL |
| Buffer | 2.0 | μL |
| Forward primer for detection of influenza (20 μM) | 1.2 | μL |
| Reverse primer for detection of influenza (20 μM) | 1.2 | μL |
| Fluorescently labeled probe for detection of influenza | 0.9 | μL |
| (10 μM) | | |
| Influenza plasmid DNA (1000 copies) | 0.625 | μL |
| Water | up to 10 | μL |

### Composition of “8×” Mixed Reagent Solution

| | | |
|---|---|---|
| Platinum Taq polymerase | 0.8 | μL |
| dNTPs (10 mM) | 1.0 | μL |
| Buffer | 2.0 | μL |
| Forward primer for detection of influenza (20 μM) | 1.6 | μL |
| Reverse primer for detection of influenza (20 μM) | 1.6 | μL |
| Fluorescently labeled probe for detection of influenza | 1.2 | μL |
| (10 μM) | | |
| Influenza plasmid DNA (1000 copies) | 0.625 | μL |
| Water | up to 10 | μL |

### Composition of “10×” Mixed Reagent Solution

| | | |
|---|---|---|
| Platinum Taq polymerase | 1.0 | μL |
| dNTPs (10 mM) | 1.25 | μL |
| Buffer | 2.0 | μL |
| Forward primer for detection of influenza (20 μM) | 2.0 | μL |
| Reverse primer for detection of influenza (20 μM) | 2.0 | μL |
| Fluorescently labeled probe for detection of influenza | 1.5 | μL |
| (10 μM) | | |
| Influenza plasmid DNA (1000 copies) | 0.625 | μL |
| Water | up to 10 | μL |

### Composition of “12×” Mixed Reagent Solution

| | | |
|---|---|---|
| Platinum Taq polymerase | 1.2 | μL |
| dNTPs (10 mM) | 1.5 | μL |
| Buffer | 2.0 | μL |
| Forward primer for detection of influenza (20 μM) | 2.4 | μL |
| Reverse primer for detection of influenza (20 μM) | 2.4 | μL |
| Fluorescently labeled probe for detection of influenza | 1.8 | μL |
| (10 μM) | | |
| Influenza plasmid DNA (1000 copies) | 0.625 | μL |
| Water | up to 10 | μL |

In the above mixed reagent solutions, as the polymerase, a product of Life Technologies, Inc. was used. As the dNTPs, a product of Roche was used. As the forward primer and the reverse primer, products of Sigma-Aldrich Co. LLC. were used. As the fluorescently labeled probe, TaqMan (registered trademark) probe manufactured by Sigma-Aldrich Co. LLC. was used. As the water, a product of Roche was used.

The composition of the buffer is as follows.

MgCl₂: 25 mM

Tris-HCl (pH 9.0): 250 mM

KCl: 125 mM

The sequences of the forward primer, the reverse primer, and the fluorescently labeled probe are as shown in the following Table 1. In Table 1, “FAM” is the reporter dye, and “BHQ1” is the quencher dye.

TABLE 1

| | |
|---|---|
| Forward primer | 5′ GAC CRA TCC TGT CAC CTC TGA |
| for detection | C 3′ |
| of InfA | (SEQ ID NO: 1) |
| Reverse primer | 5′ AGG GCA TTY TGG ACA AAK CGT |
| for detection | CTA 3′ |
| of InfA | (SEQ ID NO: 2) |
| Fluorescently | 5′ FAM-CAC AAA TCC TAA AAT TCC |
| labeled probe | CT-BHQ1 3′ |
| for detection | (SEQ ID NO: 3) |
| of InfA | |

### 4.2. Results of PCR

As a reaction mixture, 1.6 μL of the solution was extracted from each of the above mixed reagent solutions, and injected into a nucleic acid amplification reaction cartridge loaded with a silicone oil. The amount of the polymerase contained in the reaction mixture obtained from the “1×” mixed reagent solution corresponds to 0.5 U. That is, the amount of the polymerase contained in the reaction mixtures obtained from the “2×”, “4×”, “6×”, “8×”, “10×”, and “12×” mixed reagent solutions corresponds to 1 U, 2 U, 3 U, 4 U, 5 U, and 6 U, respectively.

PCR was performed for each reaction mixture using an up-and-down type PCR apparatus as the nucleic acid amplification reaction apparatus 100 by setting the first temperature (the temperature for the denaturation reaction) to 105° C., the first time (the period for the denaturation reaction) to 4 sec, the second temperature (the temperature for the annealing reaction and the elongation reaction) to 60° C., and the second time (the period for the annealing reaction and the elongation reaction) to 6 sec. Incidentally, the PCR was performed under hot start conditions by heating to 105° C. for 10 sec.

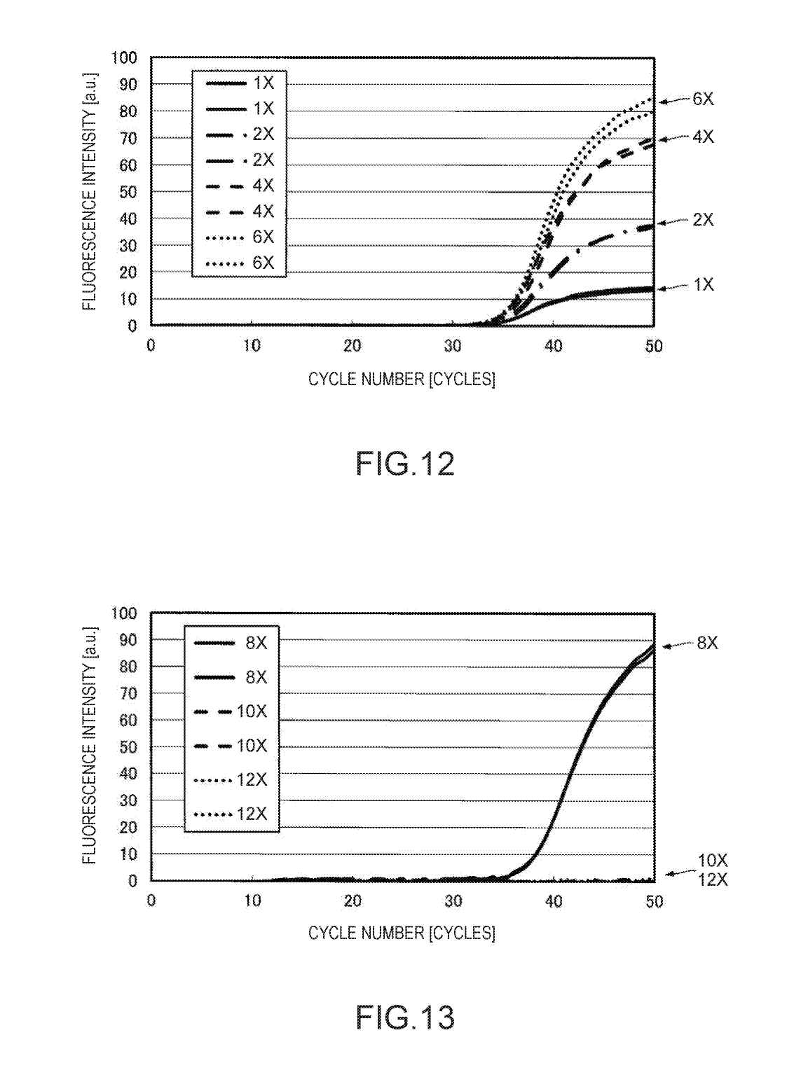

FIG. 12 is a graph showing the results of PCR using the reaction mixtures obtained from the “1×” to “6×” mixed reagent solutions. FIG. 13 is a graph showing the results of PCR using the reaction mixtures obtained from the “8×” to “12×” mixed reagent solutions. As shown in FIGS. 12 and 13, the PCR was performed twice for each reaction mixture. In FIGS. 12 and 13, the horizontal axis represents the cycle number of thermal cycles, and the vertical axis represents the fluorescence intensity measured using a fluorescence measuring device.

As shown in FIGS. 12 and 13, an increase in the fluorescence intensity could be confirmed within the range of “1×” to “8×”. The fluorescence intensity further increased within the range of “2×” to “8×”, much further increased within the range of “4×” to “8×”, and even much further increased within the range of “6×” to “8×”.

Therefore, it was found that the amplification of a nucleic acid can be detected with high sensitivity even if PCR is accelerated by setting the concentrations of the forward primer and the reverse primer contained in the reaction mixture to 0.4 μM or more and 3.2 μM or less, the amount of the polymerase contained in the reaction mixture to 0.5 U or more and 4 U or less, the concentration of the fluorescently labeled probe contained in the reaction mixture to 0.15 μM or more and 1.2 μM or less, and the concentration of the dNTPs contained in the reaction mixture to 0.125 mM or more and 1 mM or less.

In addition, it was found that the amplification of a nucleic acid can be detected with higher sensitivity even if PCR is accelerated by setting the concentrations of the forward primer and the reverse primer to 0.8 μM or more and 3.2 μM or less, the amount of the polymerase to 1 U or more and 4 U or less, the concentration of the fluorescently labeled probe to 0.3 μM or more and 1.2 μM or less, and the concentration of the dNTPs to 0.25 mM or more and 1 mM or less.

In addition, it was found that the amplification of a nucleic acid can be detected with much higher sensitivity even if PCR is accelerated by setting the concentrations of the forward primer and the reverse primer to 1.6 μM or more and 3.2 μM or less, the amount of the polymerase to 2 U or more and 4 U or less, the concentration of the fluorescently labeled probe to 0.6 μM or more and 1.2 μM or less, and the concentration of the dNTPs to 0.5 mM or more and 1 mM or less.

In addition, it was found that the amplification of a nucleic acid can be detected with even much higher sensitivity even if PCR is accelerated by setting the concentrations of the forward primer and the reverse primer to 2.4 μM or more and 3.2 μM or less, the amount of the polymerase to 3 U or more and 4 U or less, the concentration of the fluorescently labeled probe to 0.9 μM or more and 1.2 μM or less, and the concentration of the dNTPs to 0.75 mM or more and 1 mM or less.

Incidentally, the second time was set to 6 sec in the PCR in the above experimental examples, however, when the concentrations of the primers, the fluorescently labeled probe, and the dNTPs, and the amount of the polymerase were set within the above ranges, the amplification of a nucleic acid can be detected with high sensitivity as long as the second time is within the range of 1 sec to 10 sec. When the second time is set to less than 1 sec, it becomes difficult to design the nucleic acid amplification reaction apparatus or the nucleic acid amplification reaction cartridge. That is, by setting the second time to 1 sec or more, it becomes easy to design the nucleic acid amplification reaction apparatus or the nucleic acid amplification reaction cartridge, and thus, the degree of freedom of design can be increased.

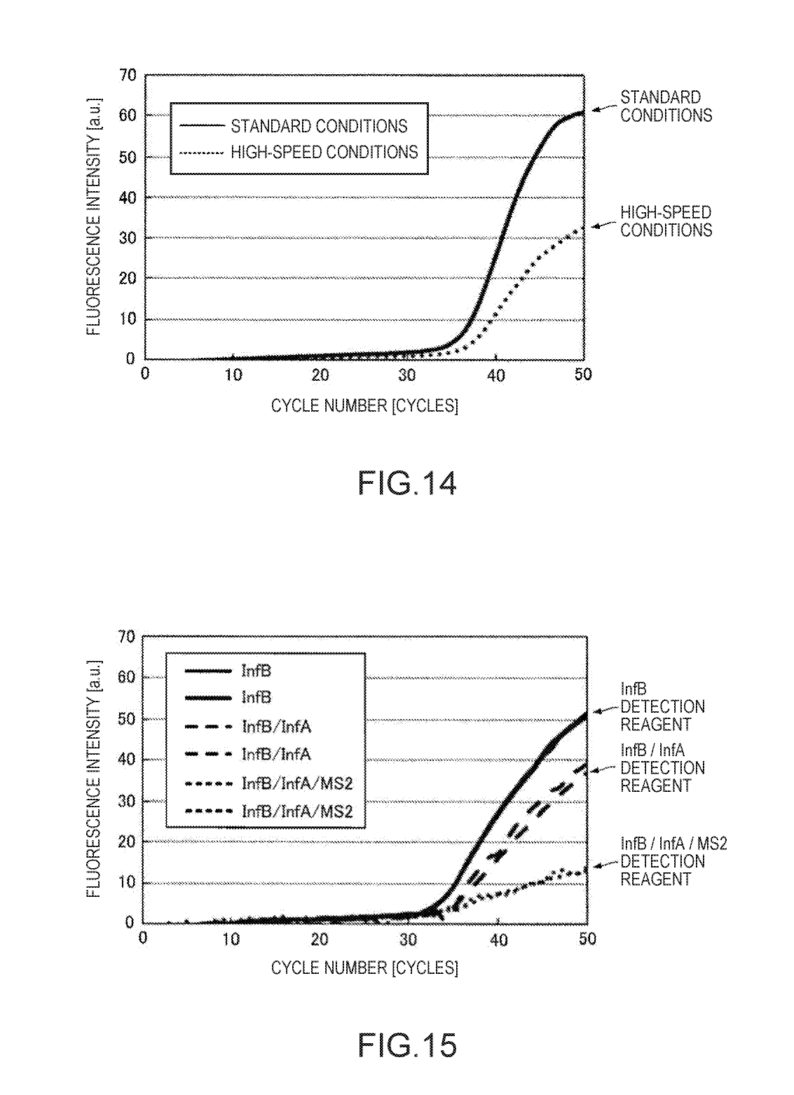

FIG. 14 is a graph showing the results of PCR under the conditions that the first time was set to 5 sec and the second time was set to 20 sec (standard conditions) and the results of PCR under the conditions that the first time was set to 3 sec and the second time was set to 6 sec (high-speed conditions).

As shown in FIG. 14, the fluorescence intensity was decreased under the high-speed conditions. This is considered to be because the nucleic acid and the fluorescently labeled probe could not sufficiently hybridize to each other under the high-speed conditions. Such a decrease in the fluorescence intensity is confirmed when the second time is set to 10 sec or less. When the concentrations of the primers, the fluorescently labeled probe, and the dNTPs, and the amount of the polymerase are set within the above ranges, the nucleic acid and the fluorescently labeled probe sufficiently hybridize to each other even if PCR is accelerated, and the amplification of the nucleic acid can be detected with high sensitivity.

FIG. 15 is a graph showing the results of multiplex PCR when the first time was set to 4 sec and the second time was set to 6 sec. In FIG. 15, PCR was performed using a reagent capable of detecting only InfB, a reagent capable of detecting InfB and InfA, or a reagent capable of detecting InfB, InfA, and bacteriophage MS2 as the nucleic acid amplification reaction reagent when 100 copies of a plasmid DNA of a type B influenza (InfB) virus were used as the template nucleic acid. As shown in FIG. 15, the PCR was performed twice for each reaction mixture.

As shown in FIG. 15, in the multiplex PCR, the fluorescence intensity was decreased. The fluorescence intensity can be increased by setting the concentrations of the primers, the fluorescently labeled probe, and the dNTPs, and the amount of the polymerase within the above ranges, and therefore, it can be said that the invention is particularly effective in the case of accelerating PCR or performing multiplex PCR.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

What is claimed is:

1. A nucleic acid amplification reaction method, comprising:
subjecting a reaction mixture containing a nucleic acid amplification reaction reagent to be used for amplifying a nucleic acid to a thermal cycle for amplifying the nucleic acid, wherein
in the thermal cycle, a heating time for an annealing reaction and an elongation reaction is 1 sec or more and 10 sec or less,
the nucleic acid amplification reaction reagent contains a forward primer, a reverse primer, a polymerase, and a fluorescently labeled probe,
the concentration of the forward primer contained in the reaction mixture is 0.4 μM or more and 3.2 μM or less,
the concentration of the reverse primer contained in the reaction mixture is 0.4 μM or more and 3.2 μM or less,
the amount of the polymerase contained in the reaction mixture is 0.5 U or more and 4 U or less, and
the concentration of the fluorescently labeled probe contained in the reaction mixture is 0.15 μM or more and 1.2 μM or less.

2. The nucleic acid amplification reaction method according to claim 1, wherein
the nucleic acid amplification reaction reagent contains dNTPs, and
the concentration of the dNTPs contained in the reaction mixture is 0.125 mM or more and 1 mM or less.

3. The nucleic acid amplification reaction method according to claim 2, wherein
the concentration of the forward primer is 0.8 μM or more and 3.2 μM or less,
the concentration of the reverse primer is 0.8 μM or more and 3.2 μM or less,
the amount of the polymerase is 1 U or more and 4 U or less,
the concentration of the fluorescently labeled probe is 0.3 μM or more and 1.2 μM or less, and
the concentration of the dNTPs is 0.25 mM or more and 1 mM or less.

4. The nucleic acid amplification reaction method according to claim 3, wherein
the concentration of the forward primer is 1.6 μM or more and 3.2 μM or less,
the concentration of the reverse primer is 1.6 μM or more and 3.2 μM or less,
the amount of the polymerase is 2 U or more and 4 U or less,
the concentration of the fluorescently labeled probe is 0.6 μM or more and 1.2 μM or less, and
the concentration of the dNTPs is 0.5 mM or more and 1 mM or less.

5. The nucleic acid amplification reaction method according to claim 4, wherein
the concentration of the forward primer is 2.4 μM or more and 3.2 μM or less,
the concentration of the reverse primer is 2.4 μM or more and 3.2 μM or less,
the amount of the polymerase is 3 U or more and 4 U or less,
the concentration of the fluorescently labeled probe is 0.9 μM or more and 1.2 μM or less, and
the concentration of the dNTPs is 0.75 mM or more and 1 mM or less.

6. A nucleic acid amplification reaction apparatus, comprising:
a mounting portion capable of mounting a nucleic acid amplification reaction cartridge which holds a reaction mixture containing a nucleic acid amplification reaction reagent to be used for amplifying a nucleic acid and a liquid having a different specific gravity from the reaction mixture and immiscible with the reaction mixture, and has a flow channel through which the reaction mixture moves;
a temperature gradient forming portion which forms a temperature gradient in the flow channel; and
a moving mechanism which moves the reaction mixture so as to subject the reaction mixture to a thermal cycle for amplifying the nucleic acid, wherein
the moving mechanism moves the reaction mixture so that a heating time for an annealing reaction and an elongation reaction in the thermal cycle is 1 sec or more and 10 sec or less,
the nucleic acid amplification reaction reagent contains a forward primer, a reverse primer, a polymerase, and a fluorescently labeled probe,
the concentration of the forward primer contained in the reaction mixture is 0.4 μM or more and 3.2 μM or less,
the concentration of the reverse primer contained in the reaction mixture is 0.4 μM or more and 3.2 μM or less,
the amount of the polymerase contained in the reaction mixture is 0.5 U or more and 4 U or less, and
the concentration of the fluorescently labeled probe contained in the reaction mixture is 0.15 μM or more and 1.2 μM or less.

7. A nucleic acid amplification reaction reagent, which is contained in a reaction mixture to be subjected to a thermal cycle for amplifying a nucleic acid, wherein
in the thermal cycle, a heating time for an annealing reaction and an elongation reaction is 1 sec or more and 10 sec or less,
the nucleic acid amplification reaction reagent contains a forward primer, a reverse primer, a polymerase, and a fluorescently labeled probe,
the concentration of the forward primer contained in the reaction mixture is 0.4 μM or more and 3.2 μM or less,
the concentration of the reverse primer contained in the reaction mixture is 0.4 μM or more and 3.2 μM or less,
the amount of the polymerase contained in the reaction mixture is 0.5 U or more and 4 U or less, and
the concentration of the fluorescently labeled probe contained in the reaction mixture is 0.15 μM or more and 1.2 μM or less.
